(19) United States
(12) Patent Application Publication
(10) Pub. No.: US 2026/0320785 A1
(43) Pub. Date: Oct. 15, 2026

(54) SYSTEM FOR DETECTING CORONARY CALCIUM

(71) Applicant: UNIVERSITA' DEGLI STUDI DI TORINO, Torino (IT)

(72) Inventors: Fabrizio D'ASCENZO, Torino (IT); Ovidio DE FILIPPO, Torino (IT); Mario IANNACCONE, Torino (IT); James Michael HUGHES, Torino (IT); Walter GROSSO MARRA, Torino (IT); Guglielmo GALLONE, Torino (IT); Paolo FONIO, Torino (IT); Attilio FIANDROTTI, Torino (IT); Marco GRANGETTO, Torino (IT); Alberto PRESTA, Torino (IT); Francesco IODICE, Torino (IT)

(21) Appl. No.: 19/483,178

(22) PCT Filed: May 9, 2024
(86) PCT No.: PCT/IB2024/054520

§ 371 Date: Nov. 10, 2025

(30) Foreign Application Priority Data
May 9, 2023 (IT) 102023000009270

(51) Int. Cl. G06T 7/00; A61B 6/00; A61B 6/50

(52) U.S. Cl. CPC G06T 7/0012; A61B 6/504; A61B 6/5217; G06T 2200/24; G06T 2207/10081; G06T 2207/10116; G06T 2207/20084; G06T 2207/30048

(57) ABSTRACT

The invention describes a system for detecting a value representative of coronary calcium (Ca_value_pred) comprising: a direct digital radiography apparatus (1) for producing a digital X-ray image (Im_Rx_P) of a patient's chest (P); a processing unit (10) to calculate the value representative of coronary calcium (Ca_value_pred) of said patient (P); a deep convolutional network (100) comprising: an encoder (110) configured to extract feature maps (F_maps), a pooling layer (115), a regressor (120) that calculates the value representative of coronary calcium (Ca_value_pred), and a binary classifier (140) that translates the continuous values produced by the regressor into binary values {0,1}.

### FIELD OF APPLICATION

The present invention relates to a system for detecting coronary calcium.

In particular, the present invention relates to a system for detecting coronary calcium on the basis of a digital X-ray image of a patient's chest.

### PRIOR ART

Primary prevention in the cardiovascular field represents an “unmet” need in cardiology, due to the difficulty of identifying patients at high risk and to problems of compliance as, above all in primary prevention, a large portion of patients do not follow the correct therapy, thus nullifying the benefits of the therapy.

The performance of diagnostic methods in the diagnosis of obstructive coronary artery disease depends on the prevalence of the disease in the studied population and, therefore, on the probability that a given patient actually suffers from coronary atherosclerosis.

For patients in whom coronary artery disease cannot be ruled out solely on the basis of a clinical assessment, non-invasive diagnostic tests are recommended in order to confirm or rule out the diagnosis and assess the risk of clinical events.

Current clinical practice guidelines recommend using cardiac computed tomography (coronary CT) as an initial test for the diagnosis of coronary atherosclerosis; coronary CT can in fact provide information about the presence of coronary calcium which can be used to improve the estimation of the probability of obstructive coronary artery disease.

For example, the absence of coronary calcium (Agatston score=0) is associated with a low prevalence of obstructive CAD (<5%).

It has been demonstrated that a quantification of coronary calcium makes it possible to classify, as compared to clinical data alone, the cardiovascular risk of patients (with an improvement in accuracy of up to 15%) and can be used to guide preventive therapy.

However, coronary CT poses several drawbacks, mainly tied to its low availability, to a non-negligible exposure to radiation and to iodised contrast media, and to the high cost.

For this reason, presently in cardiac screening a transthoracic echocardiogram, an electrocardiogram, a chest X-ray and blood tests are prescribed to the patient.

These tests make it possible to decide whether to send the patient directly to do a coronarography, i.e. an invasive examination to be performed in a hospital setting, and whether to additionally request that the patient undergo a further non-invasive test, such as, for example, coronary CT, myocardial scintigraphy, or an ergometric or stress echo test.

All these tests are complex, not rapidly performed and not widely disseminated across the territory, besides having a high cost; as a result, accessibility is difficult, which makes the operation of detecting coronary calcium inefficient and inadequate in relation to the possible therapies to be implemented, with a consequent delay in the possible diagnosis of coronary artery disease.

The object of the present invention is to provide a system for detecting coronary calcium that is not subject to the described drawbacks of the prior art, in particular to obtain a coronary calcium value on the basis of a chest X-ray via a neural network.

A particular object of the present invention is to provide a system for detecting coronary calcium that is simple to implement and use.

A particular object of the present invention is to provide a system for detecting coronary calcium that is efficient and safe and, consequently, makes it possible to provide efficient, targeted therapies.

### SUMMARY OF THE INVENTION

In a first aspect, the invention describes a system for detecting coronary calcium, according to what is described in claim 1.

Advantageous aspects are described in the dependent claims 2 to 13.

The invention provides an efficient detection tool employable in every type of hospital facility for use in everyday practice.

The invention achieves the following technical effects:
simplicity of operation and use, which makes the system employable in every setting;
efficiency and safety of detection, which, consequently, makes it possible to provide efficient, targeted therapies.

The aforementioned technical effects/advantages and other technical effects/advantages of the invention will emerge in greater detail from the description, set forth below, of an example of an embodiment given by way of non-limiting approximation with reference to the appended drawings.

### BRIEF DESCRIPTION OF THE DRAWINGS

FIG. 1 is a block diagram of the generation of a value input to a training module of the system, according to the invention.

FIG. 2 is a block diagram of a system for detecting coronary calcium, according to the invention.

FIGS. 3, 4 and 5 are specific block diagrams of blocks in FIG. 2, according to the invention.

### DETAILED DESCRIPTION

The present invention relates to a system for detecting coronary calcium which, by processing a chest X-ray, allows one to obtain a prediction of a patient's coronary calcium level through the application of machine learning techniques.

This enables an immediate definition of cardiovascular risk which can be exploited both by general practitioners and by clinical and interventional cardiologists.

The invention is based on the use of a deep convolutional neural network (DCNN) for the regression of a “calcium score” directly from a frontal chest X-ray; this value is then used to determine the presence or the absence of calcium in the coronary arteries, and whether or not the latter exceeds a predefined threshold value.

The invention describes a system for detecting a value representative of coronary calcium Ca_value_pred.

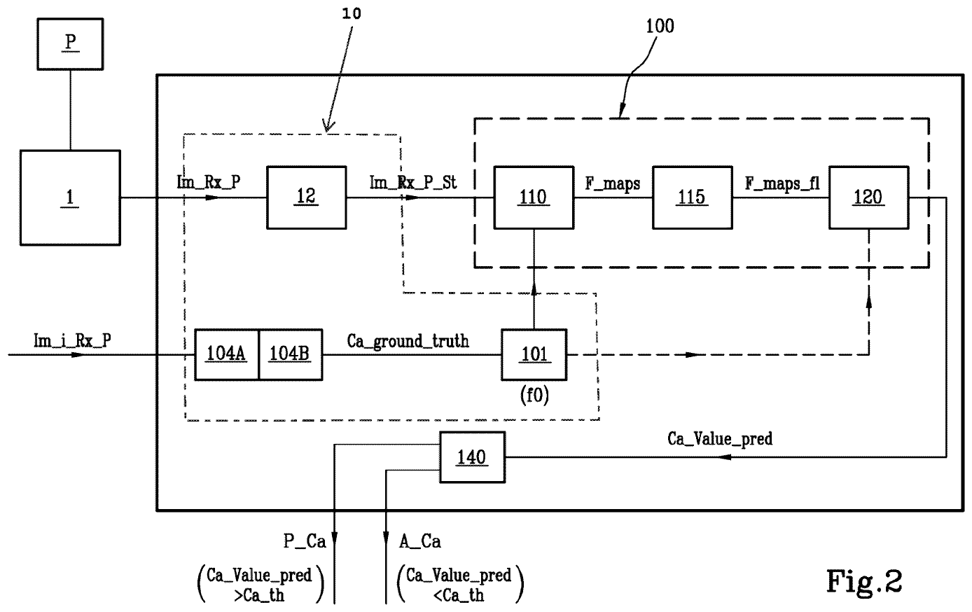

With particular reference to FIG. 2, the detection system comprises a direct digital radiography apparatus 1 adapted to produce a digital X-ray image Im_Rx_P of the chest of a patient P.

The X-ray image is acquired by means of an image acquisition system.

The image acquisition system can include one of the following:
a conventional X-ray film or a screen showing a part of the body (chest) of a patient exposed to radiation from an X-ray source which creates the graphic image. The film is developed and digitised to produce a digital X-ray image.
a direct digital device which directly produces a digital X-ray image of the patient's chest.

The digital X-ray image is processed, according to the present invention, by means of an image processing unit, described below, preferably implemented on a digital computer or digital microprocessor that can include hardware and firmware for carrying out various image processing operations.

The processed digital X-ray image is produced either as a high-resolution digital image for an electronic display or with a printer that produces a printed copy, in particular on film, of the processed X-ray image.

In general terms, the direct digital radiography apparatus 1 comprises:
an X-ray emission means X adapted to emit X rays towards the chest of the patient P;
an X-ray acquisition means adapted to receive the X rays after they have passed through the chest of the patient P;
a means for creating a graphic image on X-ray film from the acquired X rays;
a means for developing and digitising the X-ray film adapted to produce the digital X-ray image Im_Rx_P of the patient's chest.

The detection system according to the invention comprises the image processing unit, indicated by 10 in FIG. 2, configured to process the digital X-ray image Im_Rx_P and calculate the value representative of coronary calcium Ca_value_pred on the basis of the processing carried out.

In general, it should be noted that in the present context and in the claims that follow, the processing unit 10 is presented as divided into distinct functional modules (memory modules or operating modules) for the sole purpose of describing the functions thereof in a clear and complete manner.

In reality the control unit can, in one case, consist of a single electronic device, suitably programmed to perform the functions described, and the different modules can correspond to hardware entities and/or to routine software belonging to the programmed device.

Alternatively, or in addition, said functions can be performed by a plurality of electronic devices over which the aforesaid functional modules can be distributed.

The processing unit 10 can further rely on one or more processors for the execution of the instructions contained in the memory modules.

Moreover, the aforesaid functional modules can be distributed over different local or remote computers based on the architecture of the network in which they reside.

With particular reference to FIG. 2, according to the invention, the processing unit 10 comprises an operating module 12 configured to adapt the digital X-ray image Im_Rx_P so that it is optimised in content and size for a subsequent detection of the value representative of coronary calcium Ca_value_pred; the operating module 12 thereby determines a standardised digital X-ray image Im_Rx_P_St as output.

In particular, the operating module 12 is configured to execute a plurality of operating functions.

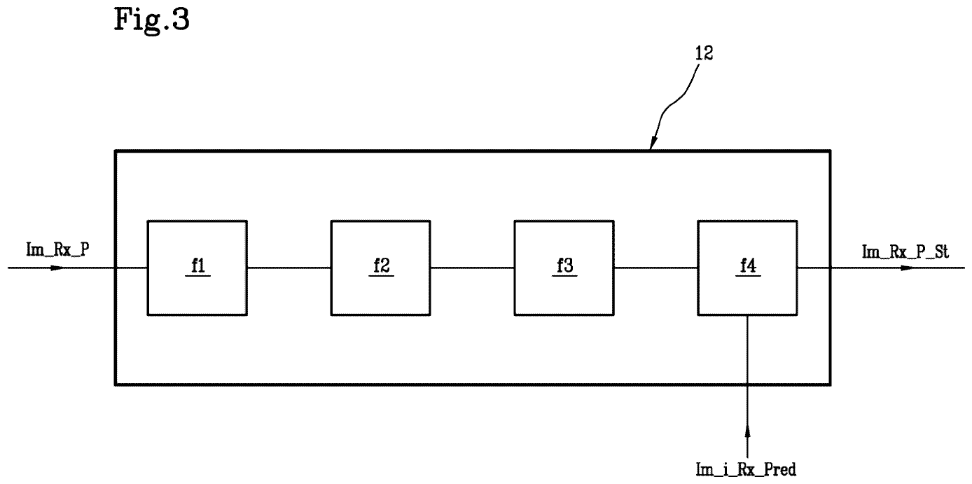

With particular reference to FIG. 3, the operating module 12 is configured to execute a conversion function on the standard 8-bit grayscale image format and a subsequent function f1 of equalisation of the histogram in order to exploit the entire dynamics available in the representation of 8-bit greyscale images.

Once this step has ended, the pixel values will be distributed in a range that goes from 0-255.

The operating module 12 is configured to execute an operational function of resizing f2 the digital X-ray image Im_Rx_P after the application of the histogram equalisation function f1.

Preferably, the image resizing is to 1248×1248 pixels, exploiting a bilinear interpolation procedure.

The operating module 12 is configured to perform an operating function of central cropping of the digital X-ray image Im_Rx_P after the resizing function f2.

Preferably, in this step a central cropping of the image is performed, thereby obtaining the final size of 1024×1024 pixels.

The technical effect achieved is to render the image size uniform.

The operating module 12 is configured to perform an operating function of standardising f4 the resized digital X-ray image Im_Rx_P on the basis of the mean and standard deviation of a set of predefined X-ray images Im_i_Rx_pred, thereby creating a standardised digital X-ray image Im_Rx_P_St.

Preferably, the set of predefined X-ray images Im_i_Rx_pred is organised like the CheXpert reference dataset.

With particular reference to FIG. 2, the system according to the invention further comprises a deep convolutional network 100.

As will be described below, the deep convolutional network 100 comprises an encoder 110 in data connection with a regressor 120 and a pooling layer 115 configured to concatenate the encoder 110 to the regressor 120. In a preferred embodiment of the invention, the architecture used to build the deep convolutional network 100 is DenseNet121, pre-trained both on ImageNet, and on the CheXpert dataset. During the training step, the weights of the encoder are frozen, with the exception of the last block, which is instead trained.

In a preferred embodiment of the invention, the DenseNet architecture is composed of 120 convolutional layers.

The deep convolutional network 100 comprises an encoder 110 in data connection with a regressor 120.

In particular, the encoder 110 is configured to extract feature maps F_maps, from the standardised digital X-ray image Im_Rx_P_St; the feature maps are representative of a synthesis of the image, i.e. representative of a positional mapping of the identifying features of the starting image.

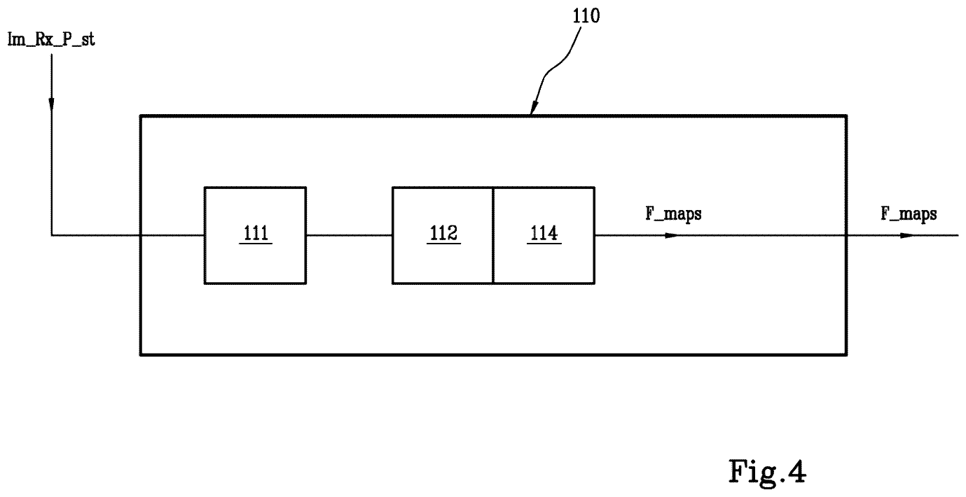

With particular reference to FIG. 4, according to the invention, the encoder 110 comprises a plurality of convolutional layers 112 and a plurality of transition layers 114 connected so that the output of one layer is directly connected to the subsequent layers, so as to concatenate the feature maps learned from the different layers.

The technical effect achieved through the learning of feature maps from different layers is an increase in the efficiency of the convolutional network and a variation with respect to the inputs.

The technical effect of the concatenation of the feature maps F_maps is the possible reuse in successive layers and reuse within the convolutional network in order to define an increasingly efficient and compact model.

Every layer of the plurality of transition layers 114 comprises convolutional layers and average pooling layers, in particular 1×1 convolutional layers and 2×2 average pooling layers.

The plurality of convolutional layers 112 and transition layers 114 is preceded by an input block 111 comprising an initial convolutional layer and a max pooling layer, in particular a 7×7 initial convolutional layer and a 2×2 max pooling layer.

The encoder 110 produces, as output from the plurality of transition layers 114, a vector of feature maps F_maps, in particular a vector of 1024 feature maps, more in particular a vector with a size of 32×32.

The encoder 110 further comprises a pooling layer 115 configured to receive the feature maps F_maps as input and to flatten them, thereby creating flattened feature maps F_maps_fl representative of the standardised digital X-ray image Im_Rx_P_St received as input to the deep convolutional network 100.

As output from the pooling layer 115, the encoder 110 produces a vector of flattened feature maps F_maps_fl.

In particular, the pooling layer 115 is configured to reduce every feature map into a single number that sums up the values obtained, using an average of the values of the preceding layers and of the feature map.

The pooling layer has the function of progressively reducing the spatial dimension of the input (width and height), so as to decrease the number of parameters and the computational load.

The neural network described according to the invention has the aim of directly extracting a calcium level Ca_value_gt from the chest X-rays.

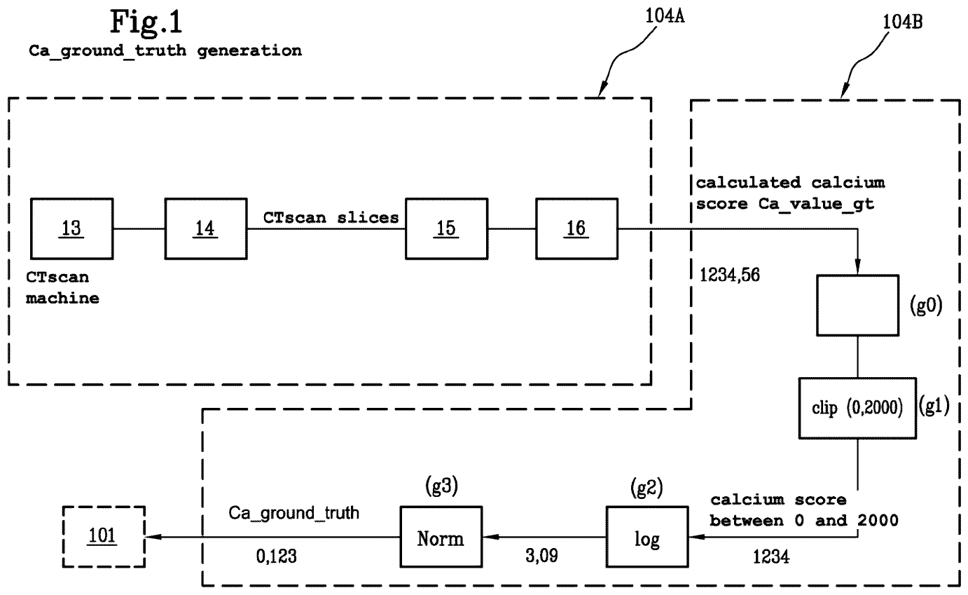

For this purpose, with particular reference to FIG. 1, the processing unit 10 comprises an extraction module 104A configured to generate a calculated calcium score value ca_value_gt.

The extraction module 104A comprises a device 13 configured to prepare a CT image, a selection interface 14 configured to select slices corresponding to a patient's heart, a selection module 15 configured to select voxels having a density that corresponds to the calcium density, a calculation module 16 configured to quantify voxels in terms of quantity and degree of calcification and configured to output the calculated calcium score value ca_value_gt.

The range of the calcium value ca_value_gt is very wide, as it goes from a minimum of 0 to a maximum of over 10000, and the calculated calcium score value ca_value_gt was re-normalised in the present invention to give greater stability to the algorithm during the training step

The application of standard solutions aimed at predicting a very wide range of values does not generally produce satisfactory results.

A specific problem with calculating values representative of coronary calcium Ca_value_pred detected by means of a digital X-ray image Im_Rx_P, is that intermediate calculated calcium score values ca_value_gt are not statically represented due to the type of incidence of the disease, which generally affects elderly subjects, with high calculated calcium score values Ca_value_gt. For this reason, a conventional system training step proves not to be efficient. This problem is not addressed in the prior art and leads to calcium score results with poor predictive value.

During the step of training the system of the invention, by contrast, the application of the convolutional neural network takes place with a suitable normalisation of the dynamic range of the calculated calcium score value ca_value_gt, i.e. with a suitable adjustment of the calculated calcium score value ca_value_gt, thus leading to an optimised result with high predictive value.

For this purpose, the processing unit 10 comprises a normalisation module 104B configured to normalise the calculated calcium score value ca_value_gt.

The normalisation module 104B, according to the invention, as also shown in FIG. 2, is directly connected to the extraction module 104A, so it uses the output value of that module, i.e. the calculated calcium score value Ca_value_gt, as the input value.

In particular, the normalisation module 104B is configured to carry out a plurality of functions:
(g0) receive as input the calculated calcium score value ca_value_gt;
(g1) cut all the calculated calcium score values higher than 2000, bringing them to a value of 2000, since discriminating among values higher than that number is not significant from a diagnostic viewpoint; in other words, the normalisation module is configured to cut off the tail of calculated calcium score values above 2000, namely the values from 2000 to 10000, flattening them at a value of 2000; in still other words, the normalisation module is configured to clip the calculated calcium score values above 2000 to a value of 2000;
(g2) transform the original domain of calcium score values through a nonlinear operation capable of compressing the dynamics of the calculated calcium score values; in particular, transforming the original domain of calcium score values into a logarithmic scale;
(g3) normalise the new calculated calcium score values using the mean and standard deviation obtained through the nonlinear operation in the new domain, in particular a logarithmic one.

The normalisation module 104B is configured to then generate as output a normalised calcium value ca_ground_truth, which will be input to the training module 101, as described below.

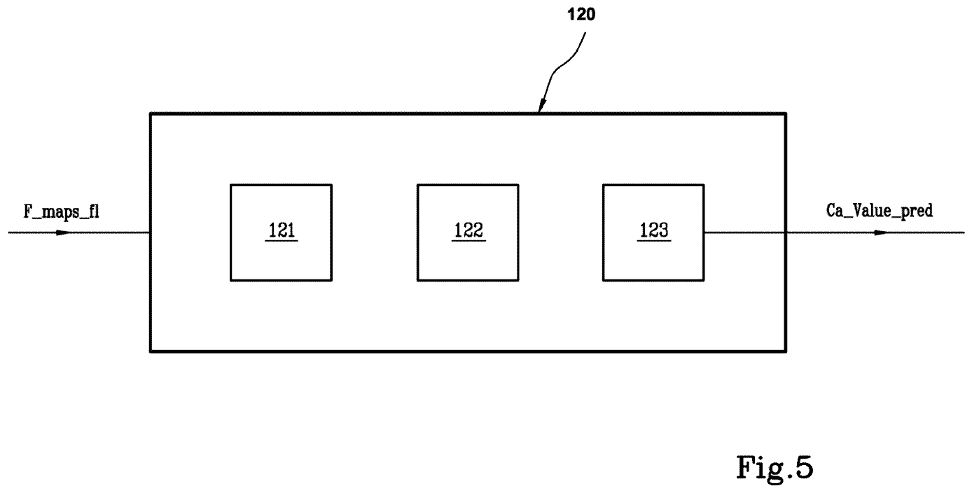

With particular reference to FIG. 5, the system according to the invention further comprises a regressor 120 configured to calculate the value representative of coronary calcium Ca_value_pred on the basis of the flattened feature maps F_maps_fl.

The regressor 120 comprises:
an input layer 121 comprising a plurality of neurons, in particular 64 neurons;
an output layer 123 with a sole final value representative of the value representative of coronary calcium Ca_value_pred;
an activation function 122 used between the input layer 121 and output layer 123, in particular represented by a rectified linear unit ReLU.

In the context of artificial neural networks, the rectified linear unit ReLU is an activation function defined as the positive part of its argument:

f(x)=x⁺=max(0, x),

where x is the input to a neuron.

With particular reference to FIG. 2, in the system according to the invention the pooling layer 115 is configured to concatenate the encoder 110 to the regressor 120.

The structure composed of the encoder 110, the pooling layer 115 and the regressor 120 allows the coronary calcium values of interest to be directly extracted from the images.

With reference to FIG. 2, the processing unit 10 further comprises a classifier 140, in data connection with the regressor 120, and configured to determine a significant presence of coronary calcium in the patient P.

The classifier 140 is such that:
if the value representative of coronary calcium Ca_value_pred calculated by the regressor 120 is higher than a threshold value ca_th, the classifier will signal a presence P_ca of coronary calcium;
if said value representative of coronary calcium Ca_value_pred calculated by said regressor 120 is lower than a threshold value ca_th, the classifier will signal an absence A_ca of coronary calcium.

The threshold value as well is expressed in a logarithmic manner, in order to be able to be used in an inference step.

With reference to FIG. 2, the processing unit 10 comprises a training module 101 configured to carry out an import function f0 to import a plurality of frontal X-rays of patients' chests Im_i_Rx_P.

The training module 101 embodies the training logic of the deep network 100: the aforesaid network is trained for 80 epochs, where in every epoch all the X-rays of the training set are passed through the network, thereby optimising the absolute error between the real calcium value, i.e. the normalised calcium value Ca_ground_truth and the predicted value, i.e. the value representative of coronary calcium ca_value_pred.

For network optimisation, use was made of the algorithm called stochastic gradient descent (SGD) with an initial learning rate of 0.001, which is decreased by a factor of 10 in epochs 20, 40, and 60. A momentum of 0.9 and weight decay of 0.0001 are also initialised (learning rate, momentum, and weight decay are all hyperparameters of SGD). A batch size of 4 images was used.

In particular, between 500 and 600, in particular 505, frontal chest X-rays were used to train the deep neural network 100,; in other words, Im_i_Rx_P preferably envisages an index value such that i=1 . . . imax, with imax comprised between 500 and 600, in particular =505.

The regressor 120 is trained by means of the training module 101 via a stochastic gradient descent (SGD) algorithm, thereby minimising the mean absolute error between the normalised real calcium value Ca_ground_truth and the predicted value, i.e. the value representative of coronary calcium Ca_value_pred.

Preferably, the network is trained for 80 epochs, using a learning rate of 0.0003 and a weight decay of 0.0001.

In an unclaimed aspect, there is envisaged a method of detecting a value representative of coronary calcium Ca_value_pred comprising the steps of:
preparing a direct digital radiography apparatus 1 to produce a digital X-ray image Im_Rx_P of the chest of a patient P;
calculating the value representative of coronary calcium Ca_value_pred of the patient P on the basis of the digital X-ray image Im_Rx_P, wherein the step of calculating comprises a sub-step of adapting the digital X-ray image Im_Rx_P so that it is optimised in content and size for a subsequent detection of the value representative of coronary calcium Ca_value_pred, thereby determining a standardised digital X-ray image Im_Rx_P_St;
preparing a deep convolutional network 100 comprising an encoder 110 configured to extract feature maps F_maps from the standardised X-ray image Im_Rx_P_St, and comprising:
   a plurality of convolutional layers 112 and a plurality of transition layers 114 so that the output of one layer is directly connected to the subsequent layers, so as to concatenate the feature maps F_maps learned from the various layers;
      wherein the encoder 110 produces a vector of the feature maps F_maps;
      wherein the encoder 110 further comprises
   a pooling layer 115 configured to flatten the feature maps F_maps, thereby creating flattened feature maps F_maps_fl representative of the standardised digital X-ray image Im_Rx_P_St;
preparing a regressor 120 to calculate the value representative of coronary calcium Ca_value_pred on the basis of the flattened feature maps F_maps_fl, comprising:
   an input layer 121 comprising a plurality of neurons, in particular 64 neurons;
   an output layer 123 with a sole final value representative of the value representative of coronary calcium Ca_value_pred;
   an activation function 122 used between the input layer 121 and the output layer 123, in particular represented by a rectified linear unit ReLU.
Preparing the pooling layer 115 configured to concatenate the encoder 110 to the regressor 120.

The method comprises further steps corresponding to the functions of the processing unit and/or the devices previously described.

A system and method for detecting coronary calcium on the basis of a digital X-ray image of a patient's chest, rather than on the basis of a CT scan of the chest, has been described.

The invention makes it possible to provide an effective detection tool employable in every type of hospital facility for use in everyday practice.

The invention achieves the following technical effects:
simplicity of operation and use, which makes the system employable in every setting;
efficiency and safety of detection, which, consequently, makes it possible to provide efficient, targeted therapies.

1. A system for detecting a value representative of coronary calcium (Ca_value_pred), comprising:
a direct digital radiography apparatus (1) adapted to produce a digital X-ray image (Im_Rx_P) of a patient's chest (P);
a processing unit (10) configured to calculate said value representative of coronary calcium (Ca_value_pred) of said patient (P) on the basis of said digital X-ray image (Im_Rx_P), wherein said processing unit (10) comprises
an operating module (12) configured to adapt said digital X-ray image (Im_Rx_P) so that it is optimised in content and size for a subsequent detection of said value representative of coronary calcium (Ca_value_pred), thereby determining a standardised digital X-ray image (Im_Rx_P_St);
a deep convolutional network (100) comprising
an encoder (110) configured to extract feature maps (F_maps) from said standardised digital X-ray image (Im_Rx_P_St) and comprising:
a plurality of convolutional layers (112) and a plurality of transition layers (114) so that the output of one layer is directly connected to the subsequent layers, so as to concatenate the feature maps (F_maps) learned from the various layers;
wherein the encoder (110) produces a vector of said feature maps (F_maps); wherein said encoder (110) further comprises
a pooling layer (115) configured to flatten the feature maps (F_maps), thereby creating flattened feature maps (F_maps_fl) representative of said standardised digital X-ray image (Im_Rx_P_St)
a regressor (120) that calculates said value representative of coronary calcium (Ca_value_pred) on the basis of said flattened feature maps (F_maps_fl), comprising:
an input layer (121) comprising a plurality of neurons;
an output layer (123) with a sole final value representative of the value representative of coronary calcium (Ca_value_pred);
an activation function (122) used between the input layer (121) and the output layer (123);
wherein said pooling layer (115) is configured to concatenate said encoder (110) to said regressor (120).

2. The system according to claim 1, wherein every layer of said plurality of transition layers (114) comprises convolutional layers and average pooling layers.

3. The system according to claim 1, wherein said plurality of convolutional layers (112) and transition layers (114) is preceded by an initial convolutional layer and a max pooling layer.

4. The system according to claim 1, wherein said operating module (12) is configured to:
execute an operational conversion function on a standard 8-bit grayscale image format and perform subsequent equalisation of a histogram (f1) in order to exploit the entire dynamics available in the representation of 8-bit greyscale images;
resize (f2) said digital X-ray image (Im_Rx_P) after the application of said histogram equalisation function (f1);
standardise (f4) said resized digital X-ray image (Im_Rx_P) on the basis of the mean and standard deviation of a set of predefined X-ray images (Im_i_Rx pred), thereby creating a standardised digital X-ray image (Im_Rx_P_St).

5. The system according to claim 4, wherein said operating module (12) is configured to perform an operating function of central cropping (f3) of the digital X-ray image (Im_Rx_P) after the resizing function (f2).

6. The system according to claim 1, wherein said processing unit (10) comprises a classifier (140) in data connection with said regressor (120) and configured to determine a significant presence of coronary calcium in said patient (P), such that:
if said value representative of coronary calcium (Ca_value_pred) calculated by said regressor (120) is higher than a threshold value (ca_th), said classifier will signal a presence (P_ca) of coronary calcium;
if said value representative of coronary calcium (Ca_value_pred) calculated by said regressor (120) is lower than a threshold value (ca_th), said classifier will signal an absence (A_ca) of coronary calcium.

7. A detection system comprising a system of claim 1, wherein said processing unit (10) comprises an extraction module (104A) configured to generate a calculated calcium score value (ca_value_gt).

8. A detection system according to claim 7, wherein an extraction model (104A) comprises:
a device (13) configured to generate a CT image;
a selection interface (14) configured to select slices corresponding to a patient's heart;
a selection module (15) configured to select voxels having a density that corresponds to the calcium density;
a calculation module (16) configured to quantify voxels in terms of quantity and degree of calcification and configured to output a calculated calcium score value (ca_value_gt).

9. A detection system according to claim 7, wherein said processing unit (10) comprises a normalisation module (104B) configured to normalise said calculated calcium value score (ca_value_gt).

10. A detection system according to claim 9, wherein said normalisation module (104B) is configured to
(g0) receive as input the calculated calcium score value (ca_value_gt);
(g1) cut all the calcium values higher than 2000, bringing them to a value of 2000;
(g2) transform the original domain of calcium values through a nonlinear operation capable of compressing the dynamic of the calculated calcium score values (ca_value_gt);
(g3) normalise the new calculated calcium score values (ca_value_gt) using the mean and standard deviation obtained through the nonlinear operation in the new domain;
said normalisation module (104B) being configured to then generate as output a normalised calcium value (ca_ground_truth) input to a training module (101).

11. A detection system according to claim 10, wherein
said transforming step (g2) is carried out through a transformation on a logarithmic scale and
said normalising step (g3) is carried out in the new logarithmic domain.

12. The detection system according to claim 10, wherein said processing unit (10) comprises said training module (101) configured to carry out an import function (f0) to import a plurality of frontal X-rays of patients' chests (Im_i_Rx_P).

13. The detection system according to claim 10, wherein said deep neural network (100) is trained by means of the training module (101) via a stochastic gradient descent (SGD) algorithm, thereby minimising the mean absolute error between the real calcium value, i.e. the normalised calcium value (Ca_ground_truth) and the predicted value, i.e. the value representative of coronary calcium (Ca_value_pred).
